(19) United States
(12) Patent Application Publication
(10) Pub. No.: US 2010/0280046 A1
(43) Pub. Date: Nov. 4, 2010

(54) Methods and Compositions for Treating Prostate Cancer, Benign Prostatic Hypertrophy, Polycystic Ovary Syndrome and Other Conditions

(75) Inventor: Timothy Andrew Stewart, San Francisco, CA (US)

Correspondence Address: CANTOR COLBURN, LLP, 20 Church Street, 22nd Floor, Hartford, CT, 06103, US

(73) Assignee: DIOBEX, INC., San Francisco, CA (US)

(21) Appl. No.: 12/160,328

(22) PCT Filed: Jan. 9, 2007
(86) PCT No.: PCT/US2007/000588

§ 371 Date: Jul. 15, 2010

Related U.S. Application Data
(60) Provisional application No. 60/758,068, filed on Jan. 10, 2006.

(51) Int. Cl. A61K 31/496; A61P 5/26; A61P 43/00; A61P 17/10; A61P 17/08; A61P 35/00; A61P 5/24

(52) U.S. Cl. 514/254.07

(57) ABSTRACT

Pharmaceutical compositions comprising the 2S,4R ketoconazole enantiomer or its pharmaceutically acceptable salts, hydrates, and solvates, that are substantially free of the 2R,4S ketoconazole enantiomer are useful to reduce testosterone synthesis and for the treatment of prostate cancer, benign prostatic hyperplasia, seborrhea, hirsutism, alopecia, polycystic ovary syndrome, and other diseases and conditions, including but not limited to systemic hyperandrogenism.

### RELATED APPLICATIONS

This application claims benefit of U.S. provisional application No. 60/758,068, filed Jan. 10, 2006, the entire contents of which is herein incorporated by reference.

### FIELD OF THE INVENTION

The present invention relates to pharmaceutical compositions and methods for treating prostate cancer and other conditions, including benign prostatic hypertrophy, polycystic ovary syndrome, androgenic alopecia, hirsutism, chronic renal failure and other conditions that can be treated by reducing testosterone synthesis. The invention therefore relates to the fields of chemistry, biology, pharmacology, and medicine.

### BACKGROUND OF THE INVENTION

Ketoconazole, 1-acetyl-4-[4-[[2-(2,4-dichlorophenyl)-2-[(1H-imidazol-1-yl)-methyl]-1,3-dioxolan-4-yl]methoxy]phenyl]piperazine, is a racemic mixture of the cis enantiomers (−)-(2S,4R) and (+)-(2R,4S) marketed as an anti-fungal agent. Ketoconazole inhibits fungal growth through the inhibition of ergosterol synthesis. Ergosterol is a key component of fungal cell walls.

More recently, ketoconazole was found to decrease plasma testosterone and to be useful, alone and in combination with other agents, in the treatment of a variety of diseases and conditions, including prostate cancer and polycystic ovary syndrome and other medical conditions that are associated with elevated testosterone levels. Testosterone is a sex steroid hormone secreted primarily from the testes. Testicular testosterone synthesis occurs in the Leydig cells and is controlled in large part by pituitary derived leutenizing hormone (LH). Androgens (the class of male sex steroid hormones that includes androstenedione, testosterone and dihydrotestosterone) are also synthesized by the adrenal glands and kidneys in both men and women. Androstenedione is converted to testosterone by the enzyme 17β dehydrogenase and testosterone can be further metabolized to the more active androgen dihydrotestosterone. These secondary steps can occur in secondary target tissues such as skin and kidney.

Testosterone circulates in the bloodstream and activates specific intracellular receptors, such as the androgen receptor (AR). Testosterone is known to be associated with numerous human diseases, including prostate cancer, benign prostatic hyperplasia, polycystic ovary syndrome, and androgen dependent alopecia.

Ketoconazole is known to inhibit some of the enzymatic steps in testosterone synthesis, such as, for example, 17α hydroxylase (Wachall et al., “Imidazole substituted biphenyls: a new class of highly potent and in vivo active inhibitors of P450 17 as potential therapeutics for treatment of prostate cancer.” Bioorg Med Chem 1999; 7(9): 1913-24, incorporated herein by reference) and 17,20 lyase (Ideyama, Y., M. Kudoh, et al. (1999). “YM116, 2-(1H-imidazol-4-ylmethyl)-9H-carbazole, decreases adrenal androgen synthesis by inhibiting C17-20 lyase activity in NCI-H295 human adrenocortical carcinoma cells.” Jpn J Pharmacol 79(2): 213-220). In addition it is known that the 2S,4R enantiomer of ketoconazole is more potent toward 17,20 lyase than is the other enantiomer 2R,4S (Rotstein, D. M., D. J. Kertesz, et al. (1992). “Stereoisomers of ketoconazole: preparation and biological activity.” J Med Chem 35(15): 2818-25, incorporated herein by reference). However there are no reports describing the effects of the two ketoconazole enantiomers on plasma levels of testosterone, and as there are multiple enzymes involved in both the synthesis and the metabolism of testosterone, it is not possible to predict what effect the two different ketoconazole enantiomers will each have on plasma levels of the active testosterone levels in a mammal.

One of the adverse side effects of ketoconazole administration is liver toxicity. Asymptomatic liver reactions can be measured by an increase in the level of liver specific enzymes found in the serum and an increase in these enzymes has been noted in ketoconazole treated patients (Sohn, “Evaluation of ketoconazole.” Clin Pharm 1982; 1(3): 217-24, and Janssen and Symoens, “Hepatic reactions during ketoconazole treatment.” Am J Med 1983; 74(1B): 80-5, each of which is incorporated herein by reference). In addition 1:12,000 patients will have more severe liver failure (Smith and Henry, “Ketoconazole: an orally effective antifungal agent. Mechanism of action, pharmacology, clinical efficacy and adverse effects.” Pharmacotherapy 1984; 4(4): 199-204, incorporated herein by reference). In rabbits the extent of liver damage correlates with the level of drug that the hepatocytes are exposed to (Ma et al., “Hepatotoxicity and toxicokinetics of ketoconazole in rabbits.” Acta Pharmacol Sin 2003; 24(8): 778-782 incorporated herein by reference), and increased hepatic exposure to the drug is believed to increase the frequency of liver damage reported in ketoconazole treated patients.

Additionally, U.S. Pat. No. 6,040,307, incorporated herein by reference, reports that the 2S,4R enantiomer is efficacious in treating fungal infections, but provides no data in support of that assertion, and also reports studies on isolated guinea pig hearts that show that the administration of racemic ketoconazole may be associated with an increased risk of cardiac arrhythmia. However, as disclosed in that patent, arrhythmia had not been previously reported as a side effect of systemic racemic ketoconazole, although a particular subtype of arrhythmia, torsades de pointes, has been reported when racemic ketoconazole was administered concurrently with terfenadine. Furthermore several published reports (for example, Morganroth, J., W. H. Lyness, et al. (1997). Lack of effect of azelastine and ketoconazole coadministration on electrocardiographic parameters in healthy volunteers. J Clin Pharmacol. 37(11): 1065-72) have demonstrated that ketoconazole does not increase the QTc interval. This interval is used as a surrogate marker to determine whether drugs have the potential for inducing arrhythmia. U.S. Pat. No. 6,040,307 also makes reference to diminished hepatoxicity associated with the 2S,4R enantiomer but provides no data in support of that assertion. The method provided in U.S. Pat. No. 6,040,307 does not allow for the assessment of hepatoxicity as the method uses microsomes isolated from frozen tissue.

Thus, there remains a need for new therapeutic agents and methods for treating diseases and conditions associated with elevated testosterone levels or activity or that may be treated by lowering testosterone level or activity that are as effective as ketoconazole but do not present, or present to a lesser degree, the issues of drug interactions and adverse side effects of ketoconazole. The present invention meets these and other needs.

### SUMMARY OF THE INVENTION

The present invention arises, in part, from the discoveries that the 2S,4R enantiomer is more effective per weight unit than racemic ketoconazole or the 2R,4S enantiomer (the other enantiomer in the racemate) at reducing the concentration of testosterone in the plasma, that the 2S,4R enantiomer is less efficiently extracted into the liver with a consequently reduced likelihood to produce hepatoxicity, and that compared to the 2R,4S enantiomer the 2S,4R enantiomer has a reduced impact on drug-drug interactions.

In a first aspect, the present invention provides a pharmaceutical composition comprising a pharmaceutically acceptable carrier and a therapeutically effective amount of the 2S,4R ketoconazole enantiomer substantially or entirely free of the 2R,4S ketoconazole enantiomer. An example of the inactive excipients and the primary reason for including these excipients in an orally available form of the 2S,4R enantiomer of ketoconazole is provided in the following table.

| Inactive | | | |
|---|---|---|---|
| Ingredient | Trade Name | Grade | Function |
| Silicified | Prosolv HD 90 | NF | Diluent |
| Microcrystalline | | | |
| Cellulose | | | |
| Lactose | Modified, 316 | NF | Diluent |
| Monohydrate | Fast Flo | | |
| Corn Starch | STA-Rx | NF | Disintegrant |
| Magnesium | N/A | NF | Lubricant |
| Stearate | | | |
| Colloidal | Cab-O-Sil M5P | NF | Glidant |
| Silicon Dioxide | | | |

The excipients listed in the preceding table can be combined in varying proportion with the 2S,4R enantiomer to obtain specific drug tablet and manufacturing characteristics. The drug tablet size can vary from 1 mg total weight to 1000 mg total weight, ideally from 100 mg total weight to 800 mg total weight. The proportion of the 2S,4R enantiomer present in the drug tablet can vary from 1% to 100%, ideally from 10% to 90%. An example of a 400 mg tablet with the 2S,4R enantiomer comprising 50% of the tablet weight is provided in the following table. In this example, dry blends were made with the (−) cis 2S,4R ketoconazole and the listed inactive excipients and the resulting mixture pressed as a dry blend into tablets.

| | | Tablet |
|---|---|---|
| | | Weight |
| Component | % w/w | (mg) |
| (−)cis 2S,4R Ketoconazole | 50.0 | 200 |
| Lactose Monohydrate, NF | 22.4 | 89.6 |
| Silicified Microcrystalline | 16.5 | 66.0 |
| Cellulose, NF | | |
| Corn Starch, NF | 10.0 | 40.0 |
| Colloidal Silicon Dioxide, NF) | 0.5 | 2.0 |
| Magnesium Stearate, NF | 0.6 | 2.4 |
| Total | 100.0 | 400.0 |

A drug tablet formulation for 2S,4R ketoconazole was provided in U.S. Pat. No. 6,040,307. This formulation included the active drug substance, (−) ketoconazole, lactose, cornstarch, water and magnesium stearate. Wet granules were generated with the ketoconazole, lactose, water, and corn starch, and these granules were dried in an oven prior to compressing into tablets with magnesium stearate and additional corn starch. Tablets were compressed and dried. This is a less optimal method than that described above using a dry blend process, as excess water and elevated temperatures are not introduced. Ketoconazole can undergo degradation (oxidation) (see Farhadi K and Maleki R. A new spectrophotometric method for the determination of ketoconazole based on the oxidation reactions. Analytical Sciences. 2001; 17 Supplement, i867-i869. The Japan Society for Analytical Chemistry; incorporated herein by reference), and oxidation reactions are accelerated in the presence of water and elevated temperatures.

In a second aspect, the present invention provides methods for treating diseases and conditions associated with elevated testosterone levels, production rates, and/or activity and treating other diseases and conditions that can be treated by reducing testosterone levels by administering a pharmaceutical composition containing a therapeutically effective amount of the 2S,4R ketoconazole enantiomer substantially or entirely free of the 2R,4S ketoconazole enantiomer.

### BRIEF DESCRIPTION OF THE FIGURES

FIG. 1 shows the effect of racemic ketoconazole and the effect of the two ketoconazole enantiomers 2R,4S, and 2S,4R on plasma testosterone. The figure shows that the 2S,4R enantiomer is more effective at lowering testosterone than any either racemic ketoconazole or the 2R,4S enantiomer of ketoconazole. The concentration of testosterone in the plasma of Sprague-Dawley rats was determined four hours after delivery by oral gavage of increasing dosage of the racemate or of the indicated enantiomer.

FIG. 2 shows the effect of racemic ketoconazole and the effect of the two enantiomers 2R,4S and 2S,4R on the time course of depression of plasma testosterone. The 2S,4R enantiomer is more effective at lowering testosterone than either racemic ketoconazole or the other cis enantiomer present in racemic ketoconazole (2R,4S). The concentration of testosterone in the plasma of Sprague-Dawley rats was determined at the indicated time after delivery by oral gavage of 200 mg/kg of either racemic ketoconazole or the two enantiomers (2S,4R and 2R,4S) present in racemic ketoconazole.

FIG. 3 shows the effect of racemic ketoconazole and the effect of the 2S,4R enantiomer on testosterone levels in patients treated with either of racemic ketoconazole (400 mg once per day) or the 2S,4R enantiomer of ketoconazole at either of 200 mg, 400 mg or 600 mg once per day. Testosterone was measured in the plasma of the patients at 4.00 am prior to treatment with any drug. The patients were then treated with the indicated drug daily for fourteen days and testosterone measured again at 4.00 am, approximately 6 hours after dosing with the drug. The figure shows the percentage change in the level of testosterone in the plasma after fourteen daily doses with the drug as compared to the testosterone levels measured prior to dosing with the drug.

FIG. 4 shows the level of the two enantiomers (2S,4R and 2R,4S) present in the plasma of patients exposed to 400 mg of racemic ketoconazole (a mixture of 200 mg 2S,4R and 200 mg 2R,4S). This figure demonstrates that the plasma levels of the 2R,4S enantiomer is approximately one third of the plasma concentration of the 2S,4R enantiomer.

FIG. 5 shows the level of the 2S,4R enantiomer present in the plasma of patients exposed to 200 mg of the 2S,4R enantiomer. This figure demonstrates that the plasma level of the 2S,4R enantiomer is approximately the same as the plasma level of the 2S,4R enantiomer when patients are given both 200 mg 2S,4R and 200 mg 2R,4S.

FIG. 6 shows the AUC (area under the curve) for total HMG CoA reductase activity for patients given Atorvastatin and either of placebo, racemic ketoconazole or 2S,4R ketoconazole.

### DETAILED DESCRIPTION OF THE INVENTION

The present invention provides pharmaceutical compositions comprising the 2S,4R ketoconazole enantiomer substantially or entirely free of the 2R,4S enantiomer. The present invention also provides methods for treating diseases and conditions associated with elevated testosterone levels or activity and diseases and conditions that may be medically treated by reducing testosterone levels and activity with these pharmaceutical compositions. To aid in understanding the invention, this detailed description is organized as follows. Section I describes methods for preparing the 2S,4R enantiomer, its solvates and salts, and pharmaceutical compositions comprising it. Section II describes unit dosage forms of the pharmaceutical compositions of the invention and methods for administering them. Section III describes methods for treating diseases and conditions by administration of the 2S,4R ketoconazole enantiomer and pharmaceutical compositions comprising the 2S,4R ketoconazole enantiomer substantially free of the 2R,4S enantiomer.

### I. Preparation of the 2S,4R Ketoconazole Enantiomer and Pharmaceutical Compositions Containing the 2S,4R Ketoconazole Enantiomer Substantially or Entirely Free of the 2R,4S Ketoconazole Enantiomer

As used herein, a composition containing “the 2S,4R ketoconazole enantiomer substantially or entirely free of the 2R,4S ketoconazole enantiomer” includes compositions that do not contain the 2R,4S ketoconazole enantiomer as well as compositions that contain substantially less of the 2R,4S ketoconazole enantiomer, relative to the amount of the 2S,4R enantiomer, than do racemic ketoconazole compositions currently approved for therapeutic use. Substantially free preferably means that the pharmaceutical composition has less than 2% of the 2R,4S enantiomer and more than 98% of the 2S,4R enantiomer. In one embodiment, the pharmaceutical composition has less than 10% of the 2R,4S enantiomer and more than 90% of the 2S,4R enantiomer. In another embodiment, the pharmaceutical composition has less than 20% of the 2R,4S enantiomer and more than 80% of the 2S,4R enantiomer. Compositions useful in the methods of the invention include, for example and without limitation, compositions in which the total ketoconazole content is comprised of at least 80%, or at least 90%, or at least 99%, or at least 99.5%, or at least 99.9% or greater of the 2S,4R enantiomer.

The 2S,4R enantiomer of ketoconazole may be obtained by optical resolution of racemic ketoconazole. Such resolution can be accomplished by any of a number of resolution methods well known to a person skilled in the art, including but not limited to those described in Jacques et al., “Enantiomers, Racemates and Resolutions,” Wiley, New York (1981), incorporated herein by reference. For example, the resolution may be carried out by preparative chromatography on a chiral column. Another example of a suitable resolution method is the formation of diastereomeric salts with a chiral acid such as tartaric, malic, mandelic acid or N-acetyl derivatives of amino acids, such as N-acetyl leucine, followed by recrystallization to isolate the diastereomeric salt of the desired enantiomer. Yet another method for obtaining compositions of the 2S,4R enantiomer substantially free of the 2R,4S enantiomer is a fractional crystallization of the diastereomeric salt of ketoconazole with (+)-camphor-10-sulfonic acid.

The 2S,4R enantiomer of ketoconazole can also be prepared directly by a variety of methods known to those of skill in the art. For example, the 2S,4R enantiomer can be prepared directly by transketolization reactions between 2-bromo-2′,4′-dichloroacetophenone and optically pure solketal tosylates, as described by Rotstein et al. (Rotstein et al., supra, incorporated herein by reference).

The present invention also provides a variety of pharmaceutically acceptable salts of the 2S,4R enantiomer of ketoconazole for use in the pharmaceutical compositions of the invention. The term “pharmaceutically acceptable salt” refers to salts prepared from pharmaceutically acceptable bases or acids, including inorganic or organic bases and inorganic or organic acids. Salts derived from inorganic bases include aluminium, ammonium, calcium, copper, ferric, ferrous, lithium, magnesium, manganic, manganous, potassium, sodium, and zinc salts, and the like. The ammonium, calcium, magnesium, potassium, and sodium salts, in particular, can be preferred for some pharmaceutical formulations. Salts in the solid form can exist in more than one crystal structure and can also be in the form of hydrates and polyhydrates. The solvates, and, in particular, the hydrates of the 2S,4R ketoconazole enantiomer are useful in the preparation of the pharmaceutical compositions of the present invention.

Salts derived from pharmaceutically acceptable organic bases include salts of primary, secondary and tertiary amines, substituted amines, including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine, and the like.

When the compound to be formulated is basic, salts can be prepared from pharmaceutically acceptable acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic; mandelic, methanesulfonic, mucic, nitric, pamoic, pantothenic, phosphoric, succinic, sulfuric, tartaric, and p-toluenesulfonic acid, and the like. Illustrative pharmaceutically acceptable acids include citric, hydrobromic, hydrochloric, maleic, phosphoric, sulfuric, and tartaric acids. Ketoconazole compounds are often basic, because the triazole ring is basic. The 2S,4R ketoconazole compound can be made and handled as a non-pharmaceutically acceptable salt (e.g. trifluoroacetate salts) during synthesis and then converted as described herein to a pharmaceutically acceptable salt.

Suitable pharmaceutically acceptable salts of the 2S,4R ketoconazole enantiomer include, but are not limited to, the mesylate, maleate, fumarate, tartrate, hydrochloride, hydrobromide, esylate, p-toluenesulfonate, benzoate, acetate, phosphate, and sulfate salts. For the preparation of pharmaceutically acceptable acid addition salts of the compound of 2S,4R ketoconazole, the free base can be reacted with the desired acids in the presence of a suitable solvent by conventional methods. Similarly, an acid addition salt can be converted to the free base form by methods known to those of skill in the art.

Pharmaceutical compositions of the invention can include metabolites of the 2S,4R ketoconazole enantiomer that are therapeutically active or prodrugs of the enantiomer. Prodrugs are compounds that are converted to therapeutically active compounds as they are being administered to a patient or after they have been administered to a patient.

Thus, the pharmaceutical compositions of the invention comprise the 2S,4R ketoconazole enantiomer, or a pharmaceutically acceptable salt, hydrate or solvate thereof, or a prodrug or active metabolite thereof, in combination with a pharmaceutically acceptable carrier and substantially or entirely free of the 2R,4S enantiomer. In one embodiment, the pharmaceutical composition contains a therapeutically effective amount of the 2S,4R enantiomer of ketoconazole or a pharmaceutically acceptable salt thereof and a pharmaceutically acceptable carrier. As noted above, pharmaceutically acceptable salts of the 2S,4R enantiomer useful in such compositions include, but are not limited to, the hydrochloride, phosphate, maleate, fumarate, tartrate, mesylate, esylate, and sulfate salts.

The “therapeutically effective amount” of the 2S,4R enantiomer of ketoconazole or pharmaceutically acceptable salt thereof will depend on the condition to be treated, the route and duration of administration, the physical attributes of the patient, including weight and other medications taken concurrently, and may be determined according to methods well known to those skilled in the art in light of the present disclosure (see Section II, below). The pharmaceutical compositions of the invention can be conveniently prepared in unit dosage form by methods well-known in the art of pharmacy as medicaments to be administered orally, parenterally (including subcutaneous, intramuscular, and intravenous administration), ocularly (ophthalmic administration), rectally, pulmonarily (nasal or oral inhalation), topically, transdermally or via buccal transfer.

The pharmaceutical compositions of the invention can be prepared by combining the 2S,4R ketoconazole enantiomer with a selected pharmaceutical carrier according to conventional pharmaceutical compounding techniques. Carriers take a wide variety of forms. For example, carriers for oral liquid compositions include, e.g., water, glycols, oils, alcohols, flavoring agents, preservatives, coloring agents and other components used in the manufacture of oral liquid suspensions, elixirs and solutions. Carriers such as starches, sugars and microcrystalline cellulose, diluents, granulating agents, lubricants, binders, disintegrating agents and the like are used to prepare oral solid dosage forms, e.g., powders, hard and soft capsules and tablets. Solid oral preparations are typically preferred over oral liquid preparations.

Thus, in one embodiment, the pharmaceutically acceptable carrier is a solid, and the pharmaceutical composition is a tablet for oral administration. Other suitable forms of the pharmaceutical compositions of the invention for oral administration include compressed or coated pills, dragees, sachets, hard or soft gelatin capsules, sublingual tablets, syrups and suspensions. The oral solid dosage forms may also contain a binder such as gum tragacanth, acacia, corn starch, or gelatin; excipients such as dicalcium phosphate; a disintegrating agent such as corn starch, potato starch, or alginic acid; a lubricant such as magnesium stearate; and/or a sweetening agent such as sucrose, lactose, or saccharin. Capsules may also contain a liquid carrier such as a fatty oil. Various other materials may be present to act as coatings or to modify the physical form of the dosage unit. For instance, tablets may be coated with shellac, sugar or both. Tablets may be coated by standard aqueous or nonaqueous techniques. The typical percentage of active compound in these compositions may, of course, be varied from, for example and without limitation, about 2 percent to about 60 percent on a w/w basis.

In another embodiment, the pharmaceutically acceptable carrier is a liquid, and the pharmaceutical composition is intended for oral administration. Oral liquids suitable for use in such compositions include syrups and elixirs and can contain, in addition to the active ingredient, sucrose as a sweetening agent, methyl and propylparabens as preservatives, a dye, and/or a flavoring, such as cherry or orange flavor.

In another embodiment, the present invention provides a pharmaceutical composition of the 2S,4R ketoconazole enantiomer suitable for parenteral administration. For parenteral administration, the pharmaceutical composition is typically contained in ampoules or vials and consists essentially of an aqueous or non-aqueous solution or emulsion. These compositions are typically in the form of a solution or suspension, and are typically prepared with water, and optionally include a surfactant such as hydroxypropylcellulose. Dispersions can be prepared in glycerol, liquid polyethylene glycols, and mixtures thereof in oils. Typically, preparations that are in diluted form also contain a preservative.

In another embodiment, the pharmaceutically acceptable carrier is a liquid, and the pharmaceutical composition is an injectable solution. The pharmaceutical injectable dosage forms, including aqueous solutions and dispersions and powders for the extemporaneous preparation of injectable solutions or dispersions, are also sterile and, at the time of administration, are sufficiently fluid for easy syringability. These compositions are stable under the conditions of manufacture and storage and are typically preserved. The carrier thus includes the solvent or dispersion medium containing, for example, water, ethanol, polyol (e.g. glycerol, propylene glycol and liquid polyethylene glycol), suitable mixtures thereof, and vegetable oils.

In another embodiment, the pharmaceutically acceptable carrier is a gel, and the pharmaceutical composition is provided in the form of a suppository. For rectal administration, the pharmaceutical composition is provided in a suppository, and the pharmaceutical acceptable carrier is a hydrophilic or hydrophobic vehicle. In another embodiment, the pharmaceutical composition useful in the methods of the invention is prepared for topical application, and the 2S,4R ketoconazole enantiomer is formulated as an ointment. The 2S,4R enantiomer can also be administered transdermally; suitable transdermal delivery systems are known in the art.

The pharmaceutical compositions of the invention also include sustained release compositions. Suitable sustained release compositions include those described in U.S. patent application publication Nos. 20050013834; 20030190357; and 2002055512 and PCT patent application publication Nos. WO 03011258 and 0152833, each of which is incorporated herein by reference, but containing only or substantially only the 2S,4R enantiomer.

### II. Unit Dosage Forms; Frequency and Duration of Administration

As noted above, any suitable route of administration can be employed for providing a mammal, typically a human, but mammals of veterinary importance, such as cattle, horses, pigs, sheep, dogs, and cats, can also benefit from the methods described herein, with a therapeutically effective dose of the 2S,4R enantiomer. For example, oral, rectal, topical, parenteral, ocular, pulmonary, or nasal administration can be employed. Dosage forms include tablets, troches, dispersions, suspensions, solutions, capsules, creams, ointments, aerosols and the like. In many embodiments of the treatment methods of the invention, the pharmaceutical composition is administered orally. The therapeutically effective dosage of the active ingredient varies depending on the particular compound employed (salt, solvate, prodrug, or metabolite), the mode of administration, the condition being treated, and the severity of the condition. Such dosages may be ascertained readily by a person skilled in the art in light of the disclosure herein.

When treating or preventing the diseases and conditions as described herein, satisfactory results can obtained when the 2S,4R ketoconazole enantiomer is administered at a daily dosage of from about 0.1 to about 10 milligrams (mg) per kilogram (mpk) of body weight, preferably given as a single daily dose or in divided doses about two to six times a day. For oral administration to a human adult patient, the therapeutically effective amount will generally be administered in doses in the range of 50 mg to 800 mg per dose, including but not limited to 100 mg per dose, 200 mg per dose, and 400 mg per dose, and multiple, usually consecutive daily doses will be administered in a course of treatment. The 2S,4R ketoconazole enantiomer pharmaceutical composition can be administered at different times of the day. In one embodiment the optimal therapeutic dose can be administered in the evening. In another embodiment the optimal therapeutic dose can be administered in the morning. The total daily dosage of the 2S,4R ketoconazole enantiomer thus can in one embodiment range from about 50 mg to about 2 g, and often ranges from about 100 mg to about 1.5 g, and most often ranges from about 200 mg to about 1200 mg. In the case of a typical 70 kg adult human, the total daily dose of the 2S,4R ketoconazole enantiomer can range from about 200 mg to about 1200 mg and will often range, as noted above, from about 200 mg to about 800 mg. This dosage may be adjusted to provide the optimal therapeutic response.

The solid unit dosage forms of the pharmaceutical compositions of the invention contain the 2S,4R ketoconazole enantiomer or a salt or hydrate thereof in an amount ranging from about 1 mg to about 2 g, often from about 1 mg to about 1 g, and more often from about 10 mg to about 500 mg. In the liquid pharmaceutical compositions of the invention designed for oral administration, the amount of the 2S,4R ketoconazole enantiomer can range from about 1 mg/ml to about 200 mg/ml. The therapeutically effective amount can also be an amount ranging from about 10 mg/ml to about 100 mg/ml. In one embodiment, the dose of the liquid pharmaceutical composition administered is an amount between 0.5 ml and 5.0 ml. In another embodiment, the dose is between about 1 ml and 3 ml. In the liquid pharmaceutical compositions of the invention designed for intravenous or subcutaneous administration the amount of the 2S,4R ketoconazole the amount of the 2S,4R enantiomer can range from about 10 to 1000 mg/ml and can be administered at a rate of between 0.01 to 1 ml/minute by either a subcutaneous or intravenous administration. Alternatively the amount of the 2S,4R enantiomer can range from about 0.1 mg/ml to 10 mg/ml and can be administered at a rate of between 0.001 ml/minute to 0.1 ml/minute by either subcutaneous or intravenous administration

As noted above, the pharmaceutical compositions of the invention will typically be administered for multiple consecutive days for periods ranging from one or more weeks to one, several, or many months. In one embodiment, the pharmaceutical compositions of the invention are administered for the treatment of a chronic disease, condition, or indication for treatment periods ranging from one month to twelve months. In another embodiment, the 2S,4R enantiomer is administered from one year to five years. In another embodiment, the 2S,4R enantiomer is administered from 5 years to 20 years. In another embodiment, the 2S,4R enantiomer is administered until there is remission from the disease or for the life of the patient.

The duration of administration in accordance with the methods of the invention depends on the disease or condition to be treated, the extent to which administration of the pharmaceutical composition has ameliorated the disease symptoms and conditions, and the individual patient's reaction to the treatment.

III. Methods for Treating Diseases and Conditions with the Pharmaceutical Compositions of the Invention

The methods and compositions of the invention are useful in the treatment of a variety of diseases and conditions in which the inhibition of testosterone synthesis provides therapeutic benefit. After a brief description of the inhibition of testosterone synthesis by the 2S,4R enantiomer of ketoconazole, a number of illustrative diseases and conditions susceptible to treatment in accordance with the present invention are described below.

Inhibition of Testosterone Synthesis

The 2S,4R enantiomer of ketoconazole is significantly more effective per unit weight at lowering the plasma concentration of testosterone than either racemic ketoconazole or the other enantiomer in racemic ketoconazole, the 2R,4S enantiomer. In addition, and as demonstrated in the Figures and Examples herein, and as distinct from racemic ketoconazole, the 2S,4R enantiomer does not undergo the same hepatic extraction as does the 2R,4S enantiomer and the 2S,4R enantiomer demonstrates reduced drug-drug interaction. Thus the methods of the present invention offer significant therapeutic benefit over methods involving the administration of racemic ketoconazole in the treatment of diseases and conditions associated with elevated levels or aberrant activity of testosterone and in the treatment of diseases in which a benefit can be obtained by lowering normal testosterone levels or activity.

The present invention provides methods for using the 2S,4R enantiomer of ketoconazole, a testosterone synthesis inhibitor, for the treatment, control, amelioration, prevention, delay in the onset of or reduction of the risk of developing the diseases and conditions due at least in part to testosterone and/or other androgens in a mammalian patient, such as a human patient. In one embodiment, the method involves the administration of a therapeutically effective amount of the 2S,4R ketoconazole enantiomer or a pharmaceutically acceptable salt or solvate thereof, substantially or entirely free of other ketoconazole enantiomers, to the patient suffering from the disease or condition.

Testosterone activity can contribute to a large number of diseases and conditions, including, but not limited to, prostate cancer, benign prostatic hyperplasia, polycystic ovary syndrome, and androgen dependent alopecia. These and other illustrative diseases and conditions susceptible to treatment with the compositions of the invention in accordance with the methods of the invention are described below.

Prostate Cancer and Benign Prostatic Hyperplasia

Growth of prostate tissue is androgen-dependent in benign prostatic hyperplasia (BPH) and early stage prostate cancer. Commonly, treatment of prostate cancer is based on surgery and/or radiation therapy, but these methods also have deleterious side effects and are ineffective in a significant percentage of cases. For example, radical prostatectomy is often accompanied by a period of dysfunction. Likewise, radiation therapy not only invokes acute adverse effects but at times also leads to long-term complications that can be debilitating or even life threatening, requiring more invasive treatments or hospitalization.

Cytotoxic chemotherapy is largely ineffective in treating prostate cancer. Even though recently developed cytotoxic agents (e.g., paclitaxel, docetaxel, and vinorelbine) have been shown to decrease prostate-specific antigen (PSA) and the pain secondary to cancer, the majority of studies using chemotherapy have failed to improve the duration of overall survival when compared to appropriate controls. In addition, the toxicity associated with these agents is unsuitable for treating elderly patients.

Luteinizing hormone-releasing hormone (LHRH) receptor agonists, and more recently antagonists, are widely used for treatment of hormone-sensitive prostate cancer. But the testicular atrophy and the loss of libido, muscle mass, and erectile function that results from decreased gonadotropin levels is only tolerable for life-threatening indications. Similarly, surgical castration is an alternative for decreasing serum androgens to treat advanced prostate cancer, but the loss of function which results can only be considered for life-threatening conditions.

5-alpha-reductase inhibitors, such as finasteride, that inhibit reduction of testosterone to the more active androgen 5-alpha-dihydrotestosterone (DHT) are used for the treatment of BPH. But 5-alpha-reductase inhibitors are only marginally effective in treatment of BPH and often require at least six months of treatment before efficacy may be observed. This marginal activity may be due to prostatic accumulation of active testosterone to eight times the normal level (Wright et al., “Relative Potency of Testosterone and Dihydrotestosterone in Preventing Atrophy and Apoptosis in the Prostate of the Castrated Rat”, J. Clin. Invest., 98(11):2558-2563 (1996)). Ketoconazole has been used to treat patients with prostate cancer (O'Rourke, M. E. (2003). “Ketoconazole in the treatment of prostate cancer.” Clin J Oncol Nurs 7(2): 235-6). However, as noted above, the utility of racemic ketoconazole for these indications is limited by the drug accumulation and hepatoxicity associated with ketoconazole.

Thus, there remains a need for new methods of treating prostate cancer and benign prostatic hyperplasia. The present invention meets this need. The present invention provides a method of treating prostate cancer and benign prostatic hyperplasia in a mammalian patient in need of such treatment, which method comprises administering to said patient a therapeutically effective amount of a pharmaceutical composition containing the 2S,4R enantiomer of ketoconazole substantially free of the 2R,4S enantiomer. In one embodiment, the method is used to treat prostate cancer. In another embodiment, the method is used to treat benign prostatic hyperplasia. Administration of a therapeutically effective amount of an testosterone synthesis inhibitor such as the 2S,4R ketoconazole enantiomer substantially free of the 2R,4S enantiomer is effective in treating, controlling, and ameliorating the symptoms of prostate cancer and benign prostatic hyperplasia, and administration of a therapeutically effective amount of a testosterone synthesis inhibitor such as the 2S,4R ketoconazole enantiomer substantially free of the 2R,4S enantiomer on a regular, daily basis can delay or prevent the progression of prostate cancer and benign prostatic hyperplasia.

The methods of the invention also include treatment methods in which the 2S,4R enantiomer of ketoconazole is administered in combination with another drug or therapy for prostate cancer or benign prostatic hyperplasia. Such other drugs or therapies include those specifically enumerated herein as well as other drugs or therapies that have been approved or are in the future approved by a regulatory authority for the treatment and/or amelioration of the symptoms of prostate cancer and benign prostatic hyperplasia.

Female Systemic Hyperandrogenism and Polycystic Ovary Syndrome

Female systemic hyperandrogenism is a diverse set of syndromes that includes Polycystic Ovary Syndrome (PCOS) and is associated with several etiologies that include Cushing's syndrome, ovarian tumors, and congenital abnormalities in sex steroid synthesis. Because elevated testosterone levels contribute to the clinical problems associated with systemic hyperandrogenism and polycystic ovary syndrome, inhibition of testosterone synthesis are beneficial in treating or controlling systemic hyperandrogenism and polycystic ovary syndrome. In one embodiment, the invention provides a method of treating systemic hyperandrogenism in a mammalian patient in need of such treatment, said method comprising administering to said patient a therapeutically effective amount of a pharmaceutical composition containing the 2S,4R ketoconazole enantiomer substantially free of the 2R,4S enantiomer.

Acne, Seborrhea Hirsutism, and Androgenetic Alopecia

The skin in women is able to synthesize testosterone and, in men, the skin can convert testosterone to the more active dihydrotestosterone. Androgens, acting through the androgen receptor, act on the sebaceous glands and on the hair follicles to cause inappropriate hair growth (hirsutism) or hair loss (alopecia) and act on the sebaceous gland to cause acne. Administration of an effective amount of a testosterone synthesis inhibitor such as the 2S,4R ketoconazole enantiomer substantially free of the 2R,4S enantiomer results in the reduction, amelioration, control, and/or prevention of acne, seborrhea, hirsutism, and/or androgenetic alopecia. In one embodiment, the invention provides a method of treating of acne, seborrhea, hirsutism, and/or androgenetic alopecia in a mammalian patient in need of such treatment, said method comprising administering to said patient a therapeutically effective amount of a pharmaceutical composition of the 2S,4R ketoconazole enantiomer substantially free of the 2R,4S enantiomer.

Precocious Puberty

Elevated androgens have been associated with precocious puberty in both males and females. The consequences of untreated precocious puberty include reduced final adult height in both males and females and an increased risk of developing polycystic ovary syndrome in females. Administration of an effective amount of testosterone synthesis inhibitor such as the 2S,4R ketoconazole enantiomer substantially free of the 2R,4S enantiomer results in the reduction, amelioration, control, or prevention of precocious puberty. In one embodiment, the invention provides a method of treating of precocious puberty in a mammalian patient in need of such treatment, said method comprising administering to said patient a therapeutically effective amount of a pharmaceutical composition of the 2S,4R ketoconazole enantiomer substantially free of the 2R,4S enantiomer.

Other Conditions

The invention provides a method for reducing plasma testosterone levels in a subject not diagnosed with or under treatment for a fungal infection, by administering a pharmaceutical composition comprising a pharmaceutically acceptable carrier and a therapeutically effective amount of 2S,4R ketoconazole enantiomer substantially free of the 2R,4S ketoconazole enantiomer to the subject. For example, the methods of the invention may also be used for treatment of diseases and conditions in which testosterone levels are not elevated (e.g., normal or below normal levels) but in whom therapeutic benefit can be obtained by reducing testosterone levels.

Additional Optional Subject Characteristics

In certain aspects of the invention, a patient being treated with a pharmaceutical composition comprising the 2S,4R ketoconazole enantiomer substantially free of the 2R,4S enantiomer is not diagnosed with and/or is not under treatment for a fungal infection. In certain aspects of the invention, a patient being treated with a pharmaceutical composition comprising the 2S,4R ketoconazole enantiomer substantially free of the 2R,4S enantiomer is not diagnosed with and/or is not under treatment for hypercholesterolemia. In certain aspects of the invention, a patient being treated with a pharmaceutical composition of the 2S,4R ketoconazole enantiomer substantially free of the 2R,4S enantiomer is not diagnosed with and/or is not under treatment for one or more diseases, disorders, or conditions independently selected from the following: (1) hyperglycemia, (2) low glucose tolerance, (3) insulin resistance, (4) obesity, (5) a lipid disorder, (6) dyslipidemia, (7) hyperlipidemia, (8) hypertriglyceridemia, (9) hypercholesterolemia, (10) low HDL levels, (11) high LDL levels, (12) atherosclerosis, (13) atherosclerosis and its sequelae, (14) vascular restenosis, (15) pancreatitis, (16) abdominal obesity, (17) neurodegenerative disease, (18) retinopathy, (19) nephropathy, (20) neuropathy, (21) Metabolic Syndrome, and (22) other conditions and disorders where insulin resistance is a component. In certain aspects of the invention, a patient being treated with a pharmaceutical composition comprising the 2S,4R ketoconazole enantiomer substantially free of the 2R,4S enantiomer is not diagnosed with and/or is not under treatment for a condition characterized by elevated testosterone levels. In certain aspects of the invention, a patient being treated with a pharmaceutical composition comprising the 2S,4R ketoconazole enantiomer substantially free of the 2R,4S enantiomer is not diagnosed with and/or is not under treatment for a condition for which reduction of testosterone levels has therapeutic effect.

Combination Therapies

Thus, a variety of diseases, disorders, and conditions can be treated, controlled, prevented or delayed with the pharmaceutical compositions and methods of this invention, including but not limited to: (1) prostate cancer, (2) benign prostatic hyperplasia, (3) systemic hyperandrogenism, (4) polycystic ovary syndrome, (5) acne, (6) seborrhea, (7) hirsutism, (8) androgenetic alopecia, (9) precocious puberty, and (10) other disorders where abnormal androgen activity is a component. In one embodiment, these treatment methods of the invention are practiced on a patient who concurrently receives another treatment for one or more of these conditions.

As is apparent from the Figures and the Examples provided herein, the 2S,4R enantiomer of ketoconazole has a reduced impact on the pharmacokinetics of coadministered drugs. Thus, the present invention provides for a method of co-administering drugs that are commonly co-administered with racemic ketoconazole without the risks of aberrant pharmacokinetics of the co-administered drug or racemic ketoconazole attendant to the administration of racemic ketoconazole.

The pharmaceutical compositions of the invention can be co-administered or otherwise used in combination with one or more other drugs in the treatment, prevention, suppression, or amelioration of the diseases, disorders, and conditions described herein as susceptible to therapeutic intervention in accordance with the methods of the invention. Typically, the combination of the drugs provided by the methods of the present invention is safer or more effective than either drug alone or of the non-2S,4R ketoconazole enantiomer drug in combination with racemic ketoconazole, or the combination is safer or more effective than would be expected based on the additive properties of the individual drugs. Such other drug(s) may be administered by a route and in an amount commonly used contemporaneously or sequentially with a pharmaceutical composition of the 2S,4R ketoconazole enantiomer substantially free of the 2R,4S enantiomer. When a pharmaceutical composition of the 2S,4R ketoconazole enantiomer substantially free of the 2R,4S enantiomer is used contemporaneously with one or more other drugs, a combination product containing such other drug(s) and the 2S,4R ketoconazole enantiomer can be utilized if the two active drugs can be coformulated. Combination therapy in accordance with the methods of the invention also includes therapies in which the pharmaceutical compositions useful in the methods of the invention and one or more other drugs are administered on different overlapping schedules. It is contemplated that, when used in combination with other active ingredients, the pharmaceutical compositions useful in the methods of the present invention or the other active ingredient or both may be used effectively in lower doses than when each is used alone. Accordingly, the pharmaceutical compositions useful in the methods of the present invention include those that contain one or more other active ingredients in addition to the 2S,4R ketoconazole enantiomer.

Examples of other drugs that may be administered in combination with a pharmaceutical composition of the present invention, either separately or, in some instances, the same pharmaceutical composition, include, but are not limited to:

(a) ovulatory stimulants such as clomiphene and letrozole; (b) androgen synthesis inhibitors other than 2S,4R ketoconazole such as C17-20 lyase inhibitors (for example, those disclosed in PCT patent application publication Nos. WO0200681 and WO03027095, each of which is incorporated herein by reference) and 5-alpha-reductase inhibitors (for example, those disclosed in PCT patent application publication Nos. WO03027225, and WO9427990, each of which is incorporated herein by reference); (c) GnRH agonists such as leuprolide; (d) GnRH antagonists (for example, such as those disclosed in PCT application Nos. WO9721435 and WO2005113516, each of which is incorporated herein by reference); (e) insulin sensitizers including (i) PPARγ agonists such as the glitazones (e.g. pioglitazone, rosiglitazone, and the like) and other PPAR ligands, including PPARα/γ dual agonists, such as KRP-297, and PPARα. agonists such as gemfibrozil, clofibrate, fenofibrate and bezafibrate, (ii) biguanides, such as metformin and phenformin, and (iii) protein tyrosine phosphatase-1B (PTP-1B) inhibitors; and (f) antiobesity compounds such as fenfluramine, dexfenfluramine, phentermine, sibutramine, orlistat, neuropeptide Y5 inhibitors, acarbose, CB1 receptor inverse agonists and antagonists, and β₃ adrenergic receptor agonists.

Thus, in one embodiment, the present invention provides a pharmaceutical composition that comprises: (1) a therapeutically effective amount of 2S,4R ketoconazole enantiomer substantially free of 2R,4S ketoconazole enantiomer; (2) a therapeutically effective amount of a compound selected from the group consisting of: (a) ovulatory stimulants; (b) androgen synthesis inhibitors selected from the group consisting of C17-20 lyase inhibitors and 5-alpha reductase inhibitors; (c) GnRH agonists; (d) GnRH antagonists; (e) insulin sensitizers selected from the group consisting of PPARγ agonists, other PPAR ligands, biguanides; and protein tyrosine phosphatase-1B (PTP-1B) inhibitors; and (f) antiobesity compounds; and (3) a pharmaceutically acceptable carrier.

Thus, the above pharmaceutical compositions and combination therapies include those in which the 2S,4R ketoconazole enantiomer, substantially or entirely free of the 2R,4S enantiomer, or a pharmaceutically acceptable salt, hydrate, or solvate thereof, is co-formulated or co-administered with one or more other active compounds. Non-limiting examples include combinations of the 2S,4R ketoconazole enantiomer with two or more active compounds selected from ovulatory stimulants, androgen synthesis inhibitors, GnRH agonists or antagonists, insulin sensitizers, and antiobesity compounds.

Thus, in one embodiment, the present invention provides a method of treating a condition selected from the group consisting of (1) prostate cancer, (2) benign prostatic hyperplasia, (3) polycystic ovary syndrome, (4) androgenitic alopecia, (5) hirsutism, (6) acne, (7) precocious puberty, and (8) other conditions and disorders where testosterone (or other androgens) is the or one of the causal factors, in a mammalian patient in need of such treatment, said method comprising administering to the patient therapeutically effective amounts of a pharmaceutical composition of the 2S,4R ketoconazole enantiomer substantially free of the 2R,4S enantiomer and of a compound or pharmaceutical composition comprising said compound selected from the group consisting of: (a) ovulatory stimulants such as clomiphene and letrozole; (b) androgen synthesis inhibitors other than 2S,4R ketoconazole such as C17-20 lyase inhibitors (for example, those disclosed in PCT patent application publication Nos. WO0200681 and WO03027095, each of which is incorporated herein by reference) and 5-alpha reductase inhibitors (for example, those disclosed in PCT patent application publication Nos. WO03027225, and WO9427990, each of which is incorporated herein by reference); (c) GnRH agonists such as leuprolide; (d) GnRH antagonists (for example, such as those disclosed in PCT application Nos. WO9721435 and WO2005113516, each of which is incorporated herein by reference) (e) insulin sensitizers including (i) PPARγ agonists such as the glitazones (e.g. pioglitazone, rosiglitazone, and the like) and other PPAR ligands, including PPARα/γ dual agonists, such as KRP-297, and PPARα. agonists such as gemfibrozil, clofibrate, fenofibrate and bezafibrate, (ii) biguanides, such as metformin and phenformin, and (iii) protein tyrosine phosphatase-1B (PTP-1B) inhibitors; (f) antiobesity compounds such as fenfluramine, dexfenfluramine, phentermine, sibutramine, orlistat, neuropeptide Y5 inhibitors, acarbose, CB1 receptor inverse agonists and antagonists, and β₃ adrenergic receptor agonists (g) antiprostate cancer agents such as mitoxantrone, estramustine phosphate, etoposide, paclitaxel, docetaxel, doxorubicin, vinblastine, and atrasentan.

The invention, numerous embodiments of which have been described above, may be further appreciated and understood by the Examples below, which demonstrate that the 2S,4R enantiomer is more effective than racemic ketoconazole or the 2R,4S enantiomer in the racemate at reducing the concentration of the active androgen in the plasma and does not impair (or impairs to a significantly less extent) drug metabolism as does racemic ketoconazole.

The concentration of the two enantiomers (2S,4R and 2R,4S) in the plasma of patients exposed to racemic ketoconazole has been reported (Stereoselective pharmacokinetics (PK) of oral ketoconazole in healthy subjects, Gerber et al. Abstr Intersci Conf Antimicrob Agents Chemother Intersci Conf Antimicrob Agents Chemother. 2003 September 14-17; 43: abstract no. A-1569.1). These authors noted that the plasma levels of the 2S,4R enantiomer was approximately three times greater than that of the 2R,4S enantiomer. An explanation for this difference is that when patients are given racemic ketoconazole, the 2R,4S enantiomer is more efficiently extracted into the liver than is the 2S,4R enantiomer leading to a greater concentration of the 2R,4S enantiomer within liver cells. An increased hepatic extraction and hepatocyte concentration of the 2R,4S enantiomer compared to the 2S,4R enantiomer suggests that the 2R,4S enantiomer may be more important than the 2S,4R for causing the liver reactions subsequent to treatment with racemic ketoconazole. Conversely, exposure of the patient to only the 2S,4R enantiomer may provide for a therapeutic exposure to ketoconazole with reduced risk of causing liver damage. It has not however been demonstrated whether there is a relatively low level of hepatic extraction of the 2S,4R enantiomer when the 2S,4R enantiomer is provided in the relative or absolute absence of the 2R,4S enantiomer. As we demonstrate below and in the examples, we have now shown in humans that the relatively lower hepatic extraction of the 2S,4R enantiomer also occurs when the 2S,4R enantiomer is provided independently of the 2R,4S enantiomer.

The use of ketoconazole as a therapeutic is complicated by the effect of ketoconazole on the P450 enzymes responsible for drug metabolism. Several of these P450 enzymes are inhibited by ketoconazole (Rotstein et al., supra). This inhibition leads to an alteration in the clearance of several important drugs such as Glivec (Dutreix et al., “Pharmacokinetic interaction between ketoconazole and imatinib mesylate (Glivec) in healthy subjects.” Cancer Chemother Pharmacol 2004; 54(4): 290-4) and methylprednisolone (Glynn et al., “Effects of ketoconazole on methylprednisolone pharmacokinetics and cortisol secretion.” Clin Pharmacol Ther 1986; 39(6): 654-9). As a result, the exposure of a patient to ketoconazole can increase the exposure of the patient to co-administered drugs. This increase in drug exposure (known as a drug-drug interaction) can make it difficult to provide an accurate and consistent dose of the co-administered drug to a patient.

Rotstein et al. (Rotstein et al., supra) have examined the effects of the two ketoconazole cis enantiomers on the principal P450 enzymes responsible for drug metabolism and reported “ . . . almost no selectivity was observed for the ketoconazole isomers” and, referring to drug metabolizing P450 enzymes: “[t]he 1050 values for the cis enantiomers were similar to those previously reported for racemic ketoconazole”. This report indicated that both of the cis enantiomers could contribute significantly to the drug-drug interaction problem observed with the ketoconazole racemate. Drug exposure is not however regulated solely by the CYP450 metabolizing enzymes. Enzymes that regulate transport into the plasma and into and out of the hepatocytes are also important. The relative effects of the two enantiomers on these enzymes have not been described and the independent impact of the two enantiomers on drug exposure in humans has not been investigated. As we show below in the Examples and Figures, the 2S,4R enantiomer has a significantly reduced effect on the exposure of patients to the HMG CoA reductase activity present in the drug Atorvastatin.

### EXAMPLES

### Example 1

### Measurement of Testosterone Following Dosing with Racemic Ketoconazole and the Enantiomers of Ketoconazole

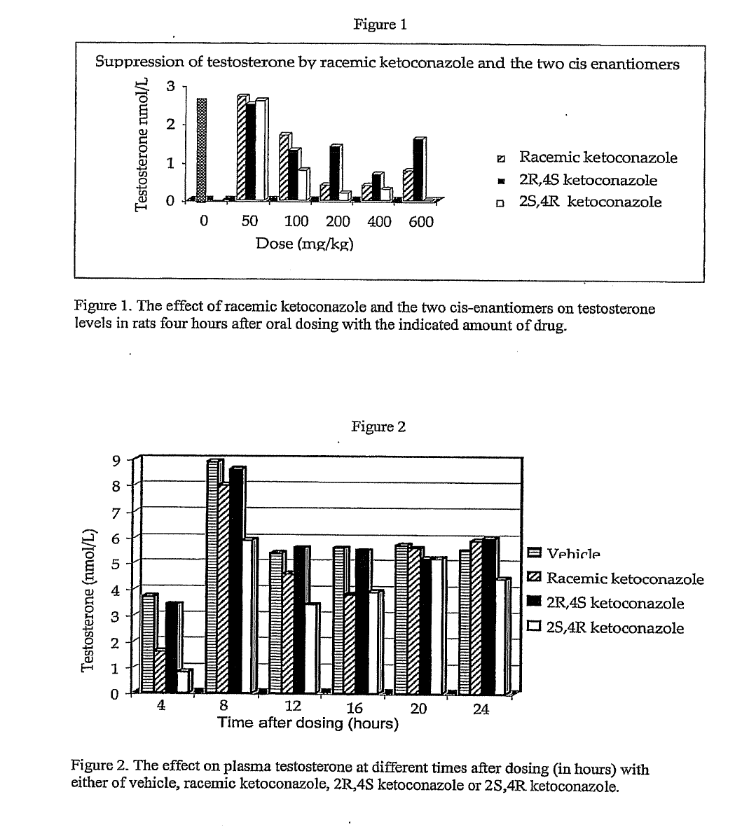

The effect of ketoconazole and the ketoconazole enantiomers on testosterone levels in the plasma of Sprague Dawley rats was determined. For the experiment described in FIG. 1, the two enantiomers and the racemic ketoconazole were suspended in olive oil. To generate the results shown in FIG. 1, fifteen groups (ten per group) of rats were used. The rats were maintained on a 14/10 hour light/dark cycle and allowed free access to food and water. Each rat was dosed with the indicated drug (or vehicle) at the indicated dosage via a gastric tube. All of the rats were dosed between 2.00 and 3.00 pm and were sacrificed four hours later (between 6.00 and 7.00 pm). Plasma was prepared and the concentration of testosterone determined by an enzyme linked immuno assay (ELISA). The results shown in FIG. 1 demonstrate that there is a dose dependent effect of both ketoconazole and the enantiomers on testosterone levels and that the 2S,4R enantiomer is significantly more efficacious than both the ketoconazole racemate and the other cis enantiomer (2R,4S).

For the experiment summarized in FIG. 2, rats (10 rats/group) were treated with racemic ketoconazole or one of the two (2S,4R and 2R,4S) cis enantiomers of ketoconazole at 200 mg/kg. The rats were maintained and dosed as described above. Plasma was prepared at the indicated times after dosing and the concentration of testosterone in the plasma determined by ELISA. The results shown in FIG. 2 demonstrate that the 2S,4R is significantly more efficacious than 2R,4S with respect to reducing testosterone levels and that testosterone levels recover following dosing with ketoconazole.

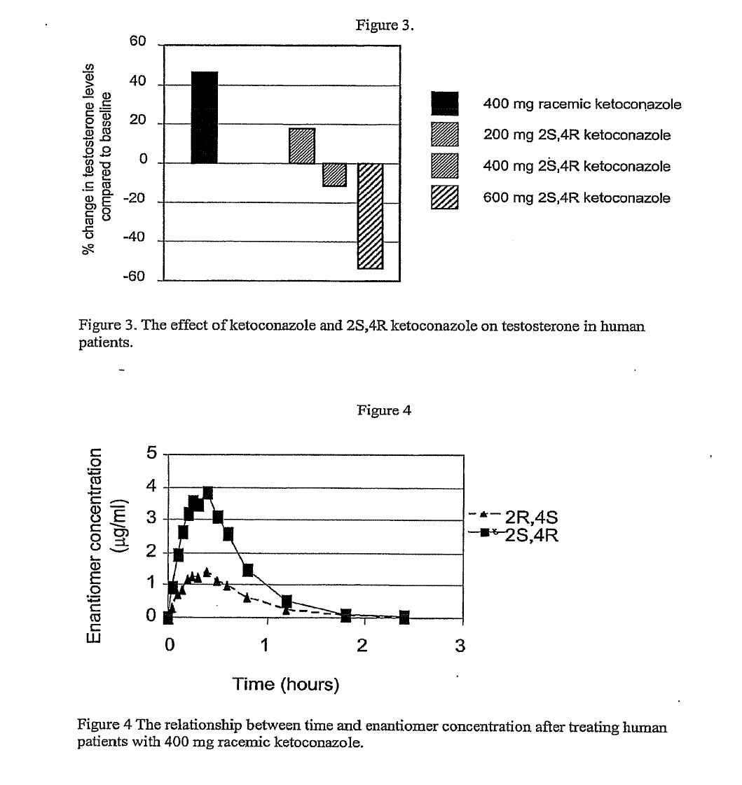

For the experiment summarized in FIG. 3, human patients were treated with either racemic ketoconazole (400 mg per day) or 2S,4R ketoconazole (200 mg, 400 mg or 600 mg/day) for fourteen days. Testosterone was measured at 4.00 am prior to the treatment with ketoconazole and again also at 4.00 am after the fourteenth daily dose of ketoconazole. The results are presented as the mean % change in testosterone levels after taking fourteen doses of the drug as compared to the testosterone levels measured prior to taking the drug.

### Example 2

### Pharmacokinetics of the Two Enantiomers Following Dosing with Racemic Ketoconazole or 2S,4R Ketoconazole

In this example, humans were treated with racemic ketoconazole or with the 2S,4R enantiomer only, and the plasma levels of the two enantiomers were determined. Patients were given either of 400 mg racemic ketoconazole comprising approximately 200 mg each of 2S,4R ketoconazole and 2R,4S ketoconazole or 200 mg of 2S,4R ketoconazole alone. Each patient was given the appropriate drug once per day in the evening. After fourteen daily doses the level of each of the two enantiomers present in the plasma of the patients was determined at the indicated time points following the last dose of drug.

As shown in FIG. 4, the pharmacokinetic profile (concentration as a function of time) of the 2S,4R enantiomer is significantly greater than the pharmacokinetic profile of the 2R,4S enantiomer after treatment of the patients with racemic ketoconazole. This difference appears to be result from a more efficient extraction of the 2R,4S enantiomer from the plasma into the liver as compared to the 2S,4R enantiomer resulting in a greater hepatic concentration of the 2R,4S enantiomer.

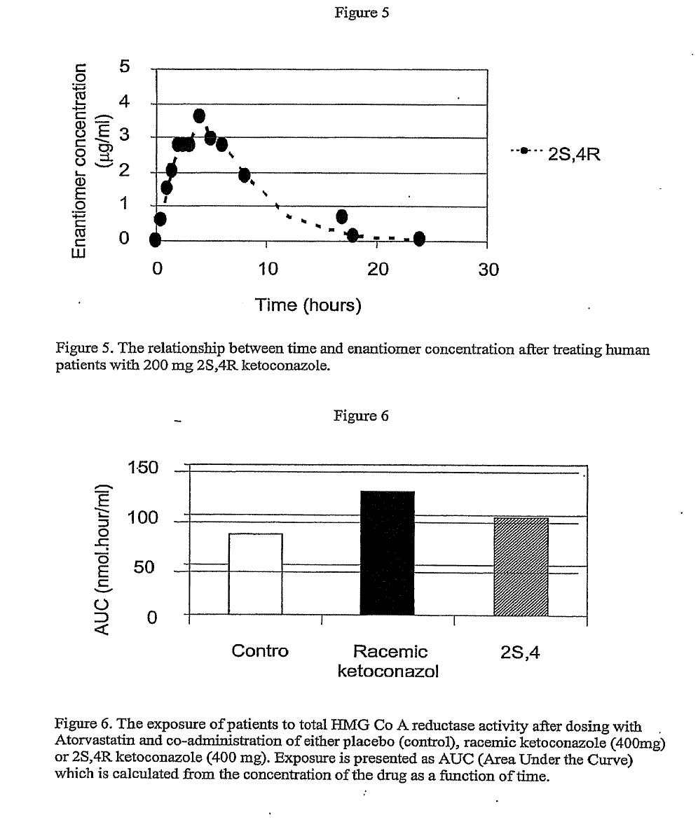

As shown in FIG. 5, the pharmacokinetic profile of the 2S,4R enantiomer is very similar when given alone (FIG. 5) or when the same amount of 2S,4R enantiomer is given simultaneously with the 2R,4S enantiomer indicating that the relatively low hepatic extraction of the 2S,4R enantiomer seen in FIG. 4 is not dependent on the presence or coadministration of the 2R,4S enantiomer

### Ketoconazole Enantiomer Assay Procedures

Assays were established and validated using isolated and purified enantiomers. Plasma from the patients treated with racemic ketoconazole, the 2S,4R enantiomer, or the vehicle control was prepared by standard methods and frozen at −70 degrees Centigrade until assayed. Solid phase extraction (SPE) and HPLC/MS/MS were used to determine the concentration of the enantiomers 2R,4S and 2S,4R, of Ketoconzaole from human plasma. 2R,4S and 2S,4R Ketoconzaole were extracted from the plasma sample using SPE, 96 well plates packed with a reversed phase C8 extraction beds from Varian (Lake Forest, Calif.). An aliquot of the extract was injected onto a HPLC/MS/MS triple quadrupole mass spectrometer (Sciex API3000). A Chiralpak AD-H, 5 μm HPLC column (2.1×150 mm) from Chiral Technologies (West Chester, Pa.) preceded by an AD-H guard column (0.4×1.0 cm) were used to separate 2R,4S and 2S,4R Ketoconzaole and the internal standard terconazole. The peak area of multiple product ions (531.4→82.2, 531.4→544.3, and 531.4→489.5) of the enantiomers of Ketoconzaole were summed and measured against the peak area of the product ion of the internal standard (terconazole, 534.5-÷ 219.3). Quantitation was performed using 1/x² weighted linear least squares regression line generated from similarly extracted plasma calibration samples ranging from 5 to 1000 ng/mL. All plasma was preserved with K₃EDTA.

### Example 3

### Measurement of Plasma Levels of HMG CoA Reductase Activity Following Co-Administration of Atorvastatin with Either of Racemic Ketoconazole or the 2S,4R Enantiomer

In this study patients were randomized in a three way cross-over protocol in which patients were treated with either of placebo, 400 mg DIO-902 or 400 mg ketoconazole for five days (Day 1-Day 5). On Day 3 the patients also received a single dose of 80 mg Atorvastatin. From Day 3 through Day 5 plasma samples were taken for the analysis of the two ketoconazole enantiomers and Atorvastatin (as well as the two biologically active metabolites (2-OH Atorvastatin and 4-OH Atorvastatin) using commercially available validated assays. The results shown in FIG. 6 are for the sum of the three active metabolites of Atorvastatin (parent Atorvastatin, 2-OH Atorvastatin and 4-OH Atorvastatin.

As shown in FIG. 6, coadministration of racemic ketoconazole with Atorvastatin results in a increase in the exposure of the patient to the sum of Atorvastatin and its active metabolites. The increase in exposure is less when the patients are coadministered 2S,4R rather than racemic ketoconazole.

The invention, having been described in detail and exemplified above, has a wide variety of embodiments; consequently, while certain embodiments of the invention have been described herein in detail, numerous alternative embodiments are contemplated as falling within the scope of the following claims.

All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same.

1. A pharmaceutical composition comprising a pharmaceutically acceptable carrier and a therapeutically effective amount of a 2S,4R ketoconazole enantiomer substantially free of a 2R,4S ketoconazole enantiomer wherein said carrier is selected from the group consisting of microcrystalline cellulose, lactose, corn starch, magnesium stearated, and colloidal silicone dioxide, and wherein said composition is prepared by a dry blend process.

2. The pharmaceutical composition of claim 1, wherein said composition does not contain any 2R,4S ketoconazole enantiomer.

3. The pharmaceutical composition of claim 1, wherein said composition contains at least two different ketoconazole enantiomers, but said 2S,4R enantiomer comprises at least 80% of total ketoconazole in said composition.

4. A method for treating a disease or condition caused at least in part by testosterone in a patient in need of such treatment, said method comprising administering a pharmaceutical composition comprising a pharmaceutically acceptable carrier and a therapeutically effective amount of a 2S,4R ketoconazole enantiomer substantially free of a 2R,4S ketoconazole enantiomer to said patient.

5. The method of claim 4, wherein said disease or condition is prostate cancer.

6. The method of claim 4, wherein said disease or condition is benign prostatic hyperplasia.

7. The method of claim 4, wherein said disease or condition is polycystic ovary syndrome.

8. The method of claim 4, wherein said disease or condition is systemic hyperandrogenism.

9. The method of claim 4, wherein said disease or condition is hirsutism.

10. The method of claim 4, wherein said disease or condition is seborrhea or acne.

11. The method of claim 4, wherein said disease or condition is precocious puberty.

12. The method of claim 4, wherein said disease or condition is selected from the group consisting of (1) prostate cancer, (2) benign prostatic hyperplasia, (3) polycystic ovary syndrome, (4) androgenetic alopecia, (5) hirsutism, (6) acne, (7) precocious puberty, and (8) other conditions and disorders where testosterone is a causal factor.

13. A method of delaying the onset of a disease or condition selected from the group consisting of (1) prostate cancer, (2) benign prostatic hyperplasia, (3) polycystic ovary syndrome, (4) androgenetic alopecia, (5) hirsutism, (6) acne, (7) precocious puberty, and (8) other conditions and disorders where testosterone is a causal factor, in a patient in need of such treatment, said method comprising administering a therapeutically effective amount of a pharmaceutical composition comprising a pharmaceutically acceptable carrier and a therapeutically effective amount of a 2S,4R ketoconazole enantiomer substantially free of a 2R,4S ketoconazole enantiomer to said patient.

14. A method of reducing the risk of developing a disease or condition selected from the group consisting of (1) prostate cancer, (2) benign prostatic hyperplasia, (3) polycystic ovary syndrome, (4) androgenetic alopecia, (5) hirsutism, (6) acne, (7) precocious puberty, and (8) other conditions and disorders where testosterone is a causal factors, in a patient in need of such treatment, said method comprising administering a therapeutically effective amount of a pharmaceutical composition comprising a pharmaceutically acceptable carrier and a therapeutically effective amount of a 2S,4R ketoconazole enantiomer substantially free of a 2R,4S ketoconazole enantiomer to said patient.

15. A method of treating a disease or condition selected from the group consisting of (1) prostate cancer, (2) benign prostatic hyperplasia, (3) polycystic ovary syndrome, (4) androgenetic alopecia, (5) hirsutism, (6) acne, (7) precocious puberty, and (8) other conditions and disorders where testosterone is a causal factor, in a patient in need of such treatment, said method comprising co-administering to said patient a therapeutically effective amount of a composition of claim 1 and a compound selected from the group consisting of: (a) an ovulatory stimulants; (b) an androgen synthesis inhibitors selected from the group consisting of C17-20 lyase inhibitors and 5-alpha reductase inhibitors; (c) a GnRH agonists; (d) an insulin sensitizers selected from the group consisting of PPARγ agonists, other PPAR ligands, biguanides; and protein tyrosine phosphatase-1B (PTP-IB) inhibitors and (e) an antiobesity compounds.

16. The method of claim 4 wherein the pharmaceutical composition of claim 1 is administered.

17. The pharmaceutical composition of claim 1, further comprising a compound selected from the group consisting of: (a) an ovulatory stimulants; (b) an androgen synthesis inhibitors selected from the group consisting of C17-20 lyase inhibitors and 5-alpha reductase inhibitors; (c) a GnRH agonists; (d) an insulin sensitizers selected from the group consisting of PPARγ agonists, other PPAR ligands, biguanides, and protein tyrosine phosphatase-1B (PTP-IB) inhibitors; and (e) an antiobesity compounds.
